(19) United States
(12) Patent Application Publication
(10) Pub. No.: US 2020/0273164 A1
(43) Pub. Date: Aug. 27, 2020

(54) BLOOD VESSELS ANALYSIS METHODOLOGY FOR THE DETECTION OF RETINA ABNORMALITIES

(71) Applicant: AEYE HEALTH LLC., New York, NY (US)

(72) Inventors: Zack DVEY-AHARON, Tel Aviv (IL); Dan MARGALIT, Ramat Gan (IL)

(21) Appl. No.: 16/757,401

(22) PCT Filed: Oct. 21, 2018
(86) PCT No.: PCT/IL2018/051124

§ 371 Date: Apr. 19, 2020

Related U.S. Application Data
(60) Provisional application No. 62/574,235, filed on Oct. 19, 2017.

(51) Int. Cl. G06T 7/00; A61B 3/12; G06T 7/13; G16H 30/40

(52) U.S. Cl. CPC G06T 7/0012; A61B 3/1241; G06T 7/13; G06T 2207/30101; G06T 2207/10024; G06T 2207/20081; G06T 2207/30041; G16H 30/40

(57) ABSTRACT

A method for detecting abnormalities or suspicious artifacts in a retinal image and training a learning machine on retinal images, the method including: acquiring data features from the retinal image; mapping a structure of a retinal blood vessel network from the retinal image; analyzing the structure to compute blood vessel features; and analyzing a set of the data features from the retinal image vis-à-vis a corresponding set of the blood vessel features. All these features are used to train a learning machine mechanism to improve statistical models.

### FIELD OF THE INVENTION

The present invention relates to methods for the detection of retina abnormalities using algorithms, machine learning and computer vision. More specifically, this invention employs computer-implemented algorithms designed to screen and diagnose conditions reflected in the retina in order to identify different findings and abnormalities related to retinal pathologies. These include diabetic retinopathy, hypertensive retinopathy, glaucoma and macular degeneration to name a few.

### BACKGROUND OF THE INVENTION

Many existing algorithms attempt to identify a variety of retinal conditions. The methodology of these algorithms involves searching for pathologies using sets of known, predefined parameters. These algorithms extract data features from retinal photos with known diagnoses and create logic or characteristics of each finding that is the target of the algorithm. The main limitation of this approach is the challenge of differentiating between different types of findings with potentially similar characteristics indicating either a serious pathology requiring immediate attention or a pathology that is negligible with only minimal clinical importance. Furthermore, findings associated with different retinal diseases may be extremely hard to distinguish from one another or from those that are harmless. Therefore, existing algorithms for the analysis of disease abnormalities may not be able to accurately detect the underlying cause of the finding.

### SUMMARY OF THE INVENTION

According to the present invention there is provided a method for detecting abnormalities or suspicious artifacts in a retinal image, the method including: acquiring data features from the retinal image; mapping a structure of a retinal blood vessel network from the retinal image; analyzing the structure to compute blood vessel features; and analyzing a set of the data features from the retinal image vis-à-vis a corresponding set of the blood vessel features.

According to further features in preferred embodiments of the invention described below retinal image is pre-processed prior to extracting the data features, for example, by at least one pre-processing method selected from the group comprising: re-sampling, normalization and contrast-limited adaptive histogram equalization.

According to still further features in the described preferred embodiments the structure of blood vessels is extracted by: (i) transforming the retinal image into a grayscale image, and (ii) applying Principal Component Analysis (PCA) to the grayscale image.

According to further features the structure of blood vessels is extracted by:

(i) applying non-linear edge detection to the retinal image. Kirsch's templates are used for the non-linear edge detection.

According to further features the structure of blood vessels is extracted by: (i) transforming the retinal image into a grayscale image, (ii) applying Principal Component Analysis (PCA) to the grayscale image, and (iii) applying non-linear edge detection to the grayscale image.

According to further features the method further includes extracting data features related to local retina properties from the retinal image.

According to further features the step of extracting data features is performed prior to the step of analyzing the extracted structure of blood vessels.

According to further features the data features are acquired by: (i) color resampling of the retinal image to 3 dimensional 30×30×30 color buckets for each 1×1, 2×2, 4×4 and 8×8 areas of the retinal image; (ii) applying transformation of color buckets to a scalar used RGB color mapping; and (iii) adding relational features to describe bucket distribution of colors in neighborhood of each the 8×8 area.

According to further features the method further includes analyzing features of the extracted structure of blood vessels vis-à-vis the extracted data features related to the local retina properties.

According to further features the method further includes feeding computed features from the analysis of the data features vis-à-vis the blood vessel features to a machine learning mechanism or statistical model.

According to another embodiment there is provided a method for training a learning machine on retinal images, the method including: mapping a structure of a blood vessel network from a retinal image; analyzing the structure to compute blood vessel features; and training the learning machine with the blood vessel features.

According to further features the method further includes acquiring data features from the retinal image; and analyzing a set of the data features vis-à-vis a corresponding set of the blood vessel features.

### BRIEF DESCRIPTION OF THE DRAWINGS

Various embodiments are herein described, by way of example only, with reference to the accompanying drawings, wherein:

FIG. 1 is a general scheme of the innovative learning mechanism that is employed for retinal imaging;

FIG. 2 is a flow chart of the stages of a typical implementation using the instant innovative methodology described herein;

FIG. 3 is an input image and a corresponding output of extracted blood vessels from the input image;

FIG. 4 is the images of FIG. 3 with the addition of two, corresponding, highlighted areas in each of the images;

FIG. 5 is a prior art pattern recognition pipeline applied to automated glaucoma detection.

### DESCRIPTION OF THE PREFERRED EMBODIMENTS

The principles and operation of the methods for the detection of retina abnormalities according to the present invention may be better understood with reference to the drawings and the accompanying description.

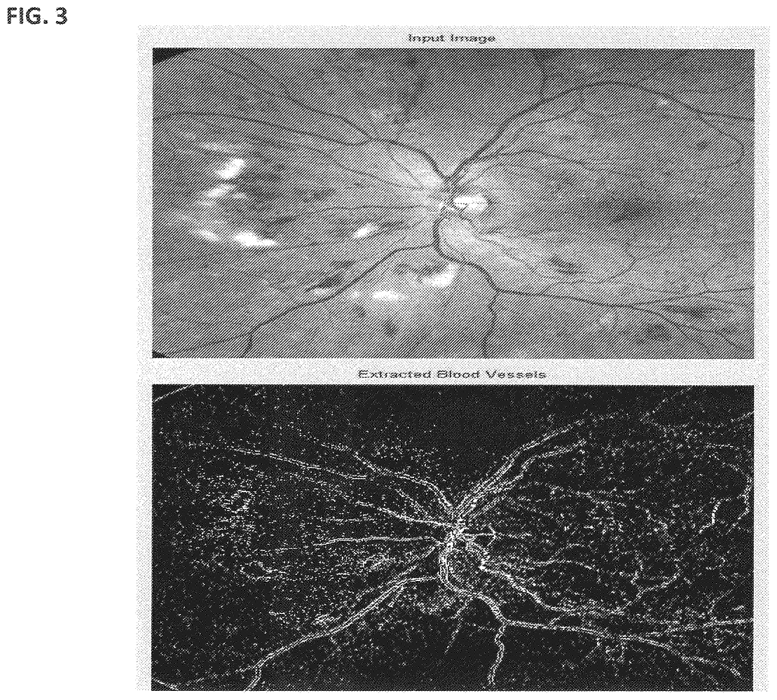

For the sake of clarity, as used herein, the terms “retina/l data features”, “common retina/l data features” and variations of the same, refer to any retinal image-related features (e.g. color, brightness, etc) that are acquired from an initial, RGB image (e.g. the input image of FIG. 3), regardless of whether the image is subjected to pre-processing or not. These features do not include data features of the structure of the retinal blood vessel network.

On the other hand, the terms “blood vessel features” or “blood vessel data features” or “data features of the blood vessel structure”, and variations thereof, refer to the data features of the extracted structure of the blood vessel network of the retina. The data also includes data gleaned from an analysis of the aforementioned data features.

The term “analysis” includes all the processes and results of examining and processing a retinal image or a map of a structure of a retinal blood vessel network. Analysis, processing and examination of the aforementioned includes, but is not limited to, extraction, computation, calculation, derivation, interpolation, extrapolation and/or other activities understood to be within the scope of meaning of the term, as understood by one skilled in the art. The term is also intended to include calculations and computations, etc. of features that result in other data features. For example, analysis of a group of pixels may identify the color features of the pixels within a given area and calculate an average or range of color for that group of pixels.

For every retinal image, the structure of the blood vessel network is identified, mapped or extracted. Both the retinal image and the mapped structure are analyzed. The retinal image is analyzed to acquire data features. The mapped structure is also analyzed to compute, acquire or extract the blood vessel features.

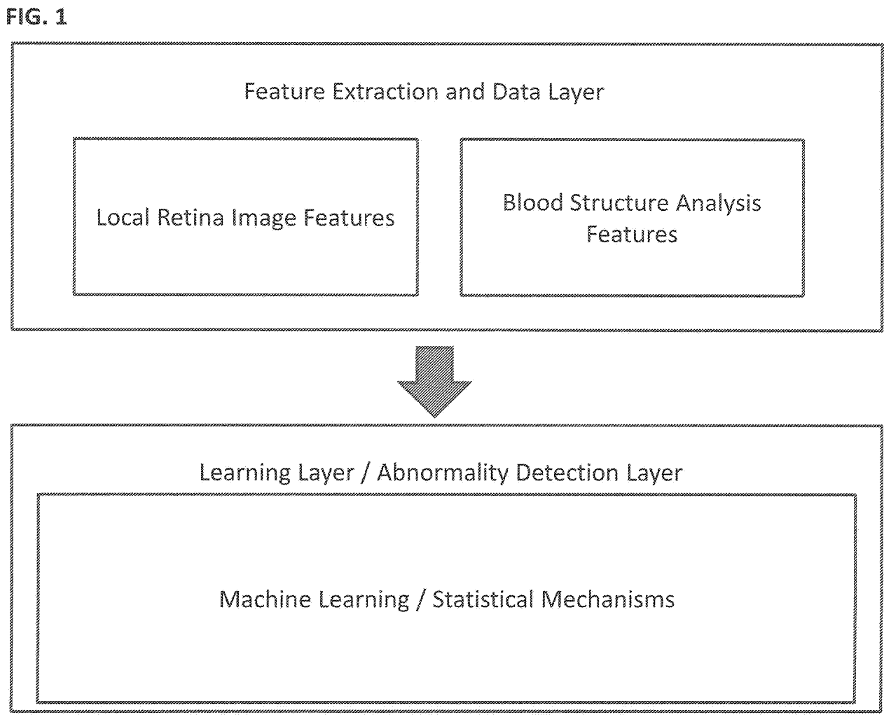

FIG. 1 illustrates a general scheme of the innovative learning mechanism that is employed for retinal imaging. The top frame represents a feature extraction mechanism and feature data layer. The feature extraction and data layer includes extraction of commonly used local retina data features as well as data features of the extracted structure of the retinal blood vessel network. Innovatively, the instant method utilizes both the retinal image data features and the blood vessel features for detecting abnormalities and training a learning machine to improve the accuracy of the statistical model used for detecting abnormalities or suspicious artifacts that may be indicative of existing or developing diseases.

In the lower frame a generic learning mechanism is presented that uses local retina data features together with the additional blood vessel data features for analytical purposes, such as, the diagnosis of retinal disease, discrimination of different findings and/or other purposes.

An innovative feature of the instant process is the addition of blood vessel structure features to a legacy digital learning mechanism algorithm. The present innovation includes an improvement to the mechanism for machine learning. Training statistical models using the instant methodology increases the accuracy of the statistical models which are then applied to new retinal images in order to detect abnormalities and/or recognize suspicious areas therein that may or may not indicate disease, or developing disease.

FIG. 3 illustrates an input image and a corresponding output of extracted blood vessels from the input image. FIG. 3 demonstrates how areas with special characteristics pertaining to the colors and shapes of a retinal image (“Input Image”) also have unique properties in a corresponding map of the blood vessels (“Extracted Blood Vessels”). Combining/cross-referencing or otherwise analyzing the two corresponding sets of properties, vis-à-vis each other, can significantly increase the scope and the level of accuracy of such analysis processes.

In conventional analysis of retinal images, regular data features are acquired from the retinal image both locally and globally. Features are acquired locally by extracting data features from each area of the image. Data features may include, for example, color, property averaging and unique background characteristics. Global data features are computed by general statistics of the whole image. For example normalization of the local data versus other images, e.g. for image brightness.

The blood vessels analysis can be done in various different ways, such as, but not limited to, using computation tables, pattern recognition techniques or visual color segmentation. The blood vessels image may also be a composition of two or more of the aforementioned processes. The resulting output (e.g. the extracted blood vessel image of FIG. 3) is a map that can be presented in various different resolutions and/or formats showing the depth and boldness of the blood vessel areas of the image, an example of which is shown in FIG. 3.

In more specific detail, the data features for the blood vessels can be both general (global) and local. An example of a local data feature is the presence of blood vessels in a predefined area or a distinguishable shape showing a high concentration, or lack, of blood vessels. A general (or global) feature could be a holistic analysis of the graph of the blood vessels. One example of global feature is the result of an analysis of the angles of veins. Another example is the comparison of the thickness of blood vessels to blood vessels in other images. Many other properties of the image can be processed into global features via analysis, computation, comparison and the like.

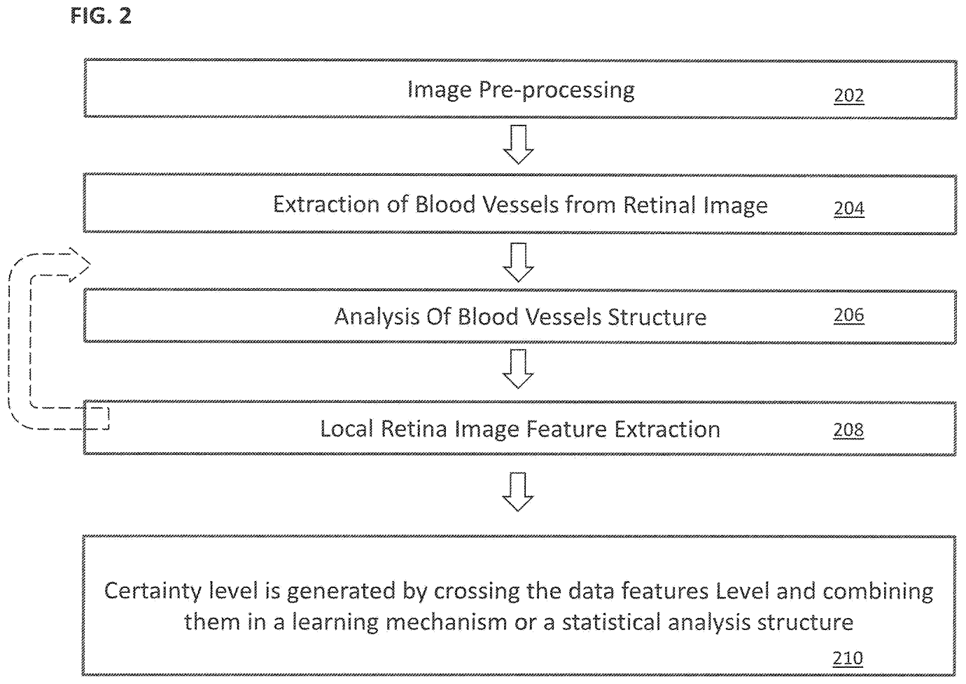

Referring now to FIG. 2, the figure illustrates a flow chart of the stages of a typical implementation using the instant innovative methodology described herein. In general, the method includes three basic steps: pre-processing the raw image, extraction and analysis of the data features and analyzing a set of data features from the retinal image vis-à-vis a corresponding set of blood vessel features.

In step 202 the method begins by pre-processing a raw image. The pre-processing phase prepares the image for the analytics phase. The pre-processing step is usually implemented when the retinal images suffer from non uniform illumination and/or poor contrast. The pre-processing step mainly includes processes of re-sampling and/or normalization of the raw images. Contrast-limited adaptive histogram equalizations can also be used to correct non uniform illumination and to improve contrast of an image. Preprocessing is usually necessary in most cases of retinal image analysis.

Images may be re-sampled to any predefined set of dimensions. Exemplarily, images are resampled to 128×128 configurations after removing external black rectangles from the image which are not relevant to the disease presence, in the case of retinal images.

The next step, step 204 is the acquisition of the structure of the blood vessels in the [pre-processed] image (theoretically, some images do not need to be pre-processed if they are in the exact format and condition required for analysis; practically this is unlikely so happen often if at all). Acquisition or extraction (also referred to herein as mapping or identifying) of the structure of the blood vessels can be implemented in various different ways. All the methods for mapping of the structure of the network of blood vessels known in the art are included within the scope of the invention. Some exemplary methods for extraction of the blood vessels are discussed below.

### Method 1

Transforming the image into a grayscale image and then applying Principal Component Analysis (PCA) (i.e. S=X\hat{ }TX or SVD) with or without heuristics on the size and/or color of the main component of the blood vessel.

### Method 2

The second exemplary method is based on non-linear edge detection, and in particular, uses Kirsch's templates (noted as finding parameters that maximize [1] below).

$${h}_{n , m} = {max}_{z = 1 , … , 8} {\sum }_{i = - 1}^{1} {\sum }_{j = - 1}^{1} {g}_{ij}^{( z )} \cdot {f}_{n + 1 , m + j} \qquad [ 1 ]$$

### Method 3

Using a combination of methods 1 and 2 described above, i.e. transforming the retinal image into a grayscale image, applying (PCA) to the grayscale image, and applying non-linear edge detection (e.g. employing Kirsch's templates) to the grayscale image. Method 3 is the most preferred method for use, but by no means the only method included within the scope of the invention, as mentioned above.

After mapping/extracting/acquiring the structure of the blood vessels from the image, in step 206, the structure of the blood vessels is analyzed. Blood vessel analysis has been discussed above, with reference to FIG. 1.

The next stage, step 208, includes acquiring and extracting [common] data features from the retinal image related to the local retina properties. Step 208 can alternatively be performed prior to the analysis of the blood vessel structure, in step 206. The instant innovative process can employ any of the methods known in the art for summarizing and retrieving local information from different areas of the retinal image.

One exemplary implementation of step 208 employs the following features:

(A) providing the original raw-data image;

(B) color resampling to 3 dimensional 30×30×30 color buckets for each 1×1, 2×2, 4×4 and 8×8 areas of the original image;

(C) a one-directional transformation of color to a scalar used RGB color mapping; and

(D) relational features added to describe bucket distribution of colors in neighborhood of each 8×8 area.

Step 210 entails analyzing a set of data features from the retinal image vis-à-vis a corresponding set of blood vessel features. The sets of data (alone, in combination, or the results of the analysis thereof) are used to train a learning machine in order to improve the statistical model/s which are later used for analyzing new retinal images. In some embodiments, step 210 relates to the way the blood vessel features are combined with the commonly acquired features of the retinal image. In other embodiments, corresponding sets of data features are cross referenced. Step 210 can be implemented in various ways. Two popular approaches are:

1. Simply add the blood vessel features (as explained above) to the learning mechanism that is employed to analyze the commonly acquired features (can be either to the very same mechanism or to a different sub-mechanism under the same structure);

2. Use the blood vessel structure data to increase the level of certainty or decrease the level suspiciousness, of other underlying phenomena.

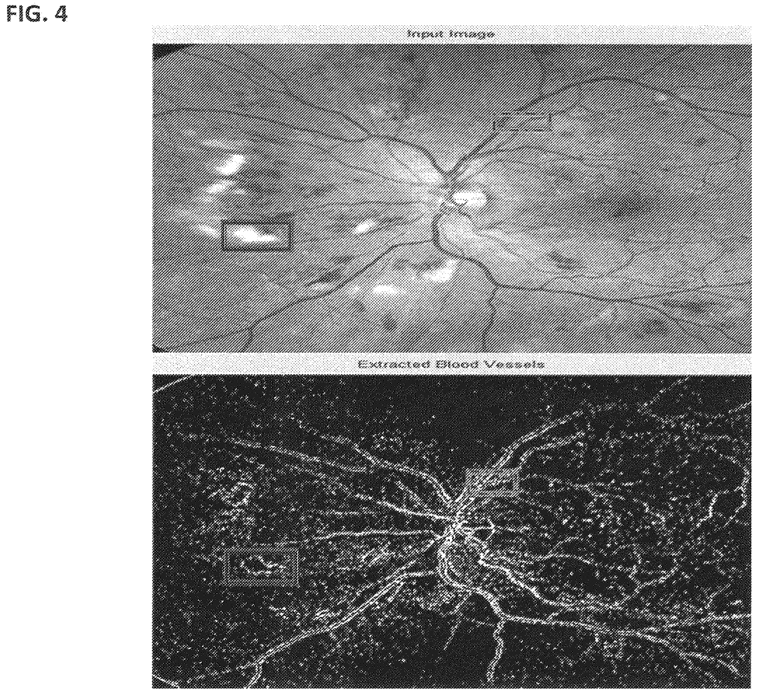

FIG. 4 illustrates the images of FIG. 3 with the addition of two, corresponding, highlighted areas in each of the images. The right-hand broken-border rectangles (in both images) highlight an area which is well populated with blood vessels. The left-hand full-border rectangles (in both images) highlight a round-shaped area. The images demonstrate how round-shaped areas with no visible blood vessels (indicating a dramatically higher potential to host a retinal disease) are clearly demarcated and identified in the extracted blood vessel map.

In a preferred, but exemplary, implementation, the Kirsch optimized value and the sum of blood vessels found are added to the coordinate features, per area block (i.e. 1×1, 2×2, 4×4 or 8×8). Blood vessel detection is also followed by circular or ellipse detection, using curve analysis. If a circular or ellipse shape is found in the area of the aforementioned ‘area block’ (even a partial circle or ellipse shape), the coordinate feature used for the analysis is the largest radius found. If no such shape is found, then the feature value is 0 (zero).

In a comparative study conducted in Israel by the authors, the above-described analysis algorithm was performed on images, with and without the addition of the blood vessels structure-related features, with the goal of detecting the specific illness of Diabetic Retinopathy. In all cases the relevant features were fed to a convolution neural network implemented in Python having 6 hidden layers between the input layer and the classifier projecting layer. The comparative analysis was performed on 750 retinal images of which 500 images were from healthy subjects and 250 images were from subjects who had previously been diagnosed with Diabetic Retinopathy (DR) by an expert.

The study found that including the blood vessel features in the analysis resulted in a 93.8% level of accuracy as opposed to 91.5% level of accuracy achieved without including the blood vessel features in the analysis. The study was performed with the leave-one-out test and validation set-up. Similar improvement of accuracy and contribution to P-value (in leave-p-out cross validation) was also evident in a different configuration and amount of input data.

Summary of the study:

I. Goals

Automated detection of diabetic retinopathy.

II. Methodology Summary

A combination of (A) local retinal imagining data and (B) blood vessel information is applied; the suspicious areas of the blood vessel structure are cross-referenced with local retina texture features. Parameters are learned and adjusted using statistical models.

III. Data

750 fundus images, 250 diagnosed with DR

IV. Results

92.4% sensitivity

96.3% specificity

P-Value<1%

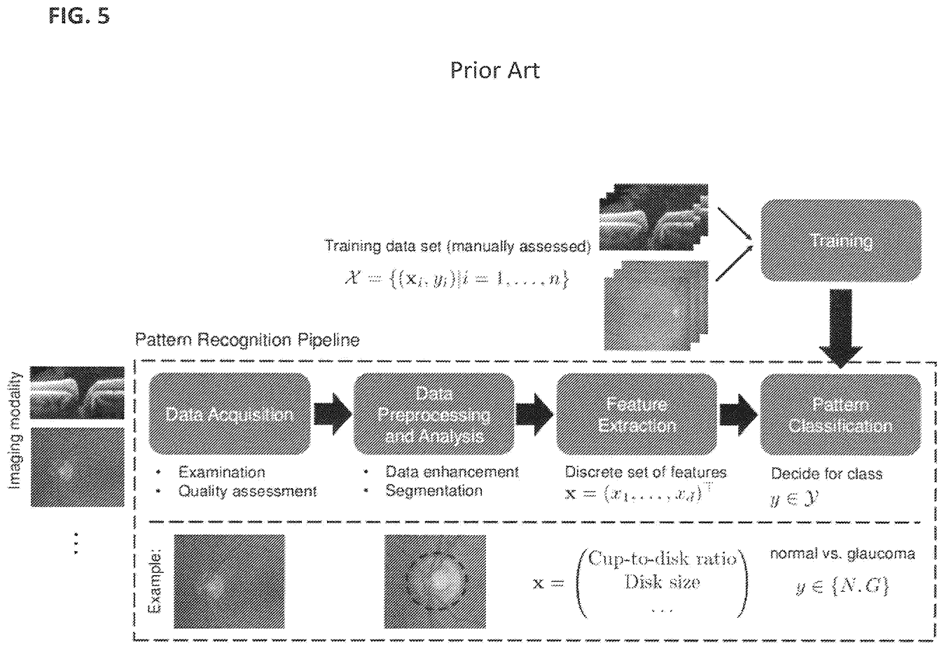

A prior art pattern recognition pipeline applied to automated glaucoma detection is depicted in FIG. 5. Retinal image data is (i) acquired with an eye imaging modality such as fundus imaging or optical coherence tomography (OCT), (ii) preprocessed and analyzed as preparation for pattern recognition techniques, (iii) used to extract relevant features to detected traces of glaucoma and (iv) used in a classification stage trained with manually classified image data. A common example workflow is visualized for glaucoma detection based on fundus photographs.

Innovatively, the instant method combines the common features of the retinal image with the data features acquired by analyzing the corresponding areas of the extracted blood vessel network structure. As discussed above, the result of combining the sets of data features is improved accuracy in anomaly detection (e.g. increase the level of certainty or decrease the level suspiciousness). The data is fed into the machine learning mechanism, improving the statistical model.

While the invention has been described with respect to a limited number of embodiments, it will be appreciated that many variations, modifications and other applications of the invention may be made. Therefore, the claimed invention as recited in the claims that follow is not limited to the embodiments described herein.

What is claimed is:

1. A method for detecting abnormalities or suspicious artifacts in a retinal image, the method comprising:
acquiring data features from the retinal image;
mapping a structure of a retinal blood vessel network from the retinal image;
analyzing said structure to compute blood vessel features; and
analyzing a set of said data features from the retinal image vis-à-vis a corresponding set of said blood vessel features.

2. The method of claim 1, wherein said retinal image is pre-processed prior to extracting said data features.

3. The method of claim 2, wherein said retinal image is pre-processed by at least one pre-processing method selected from the group comprising: re-sampling, normalization and contrast-limited adaptive histogram equalization.

4. The method of claim 1, wherein said structure of blood vessels is extracted by:
(i) transforming the retinal image into a grayscale image, and
(ii) applying Principal Component Analysis (PCA) to said grayscale image.

5. The method of claim 1, wherein said structure of blood vessels is extracted by:
(i) applying non-linear edge detection to the retinal image.

6. The method of claim 5, wherein Kirsch's templates are used for said non-linear edge detection.

7. The method of claim 1, wherein said structure of blood vessels is extracted by:
(i) transforming the retinal image into a grayscale image,
(ii) applying Principal Component Analysis (PCA) to said grayscale image, and
(iii) applying non-linear edge detection to said grayscale image.

8. The method of claim 1, further comprising:
extracting data features related to local retina properties from said retinal image.

9. The method of claim 8, wherein said step of extracting data features is performed prior to said step of analyzing said extracted structure of blood vessels.

10. The method of claim 1, wherein said data features are acquired by:
(i) color resampling of the retinal image to 3 dimensional 30×30×30 color buckets for each 1×1, 2×2, 4×4 and 8×8 areas of the retinal image;
(ii) applying transformation of color buckets to a scalar used RGB color mapping; and
(iii) adding relational features to describe bucket distribution of colors in neighborhood of each said 8×8 area.

11. The method of claim 8, further comprising:
analyzing features of said extracted structure of blood vessels vis-à-vis said extracted data features related to said local retina properties.

12. The method of claim 11, further comprising:
feeding computed features from said analysis of said data features vis-à-vis said blood vessel features to a machine learning mechanism or statistical model.

13. A method for training a learning machine on retinal images, the method comprising:
mapping a structure of a blood vessel network from a retinal image;
analyzing said structure to compute blood vessel features; and
training the learning machine with said blood vessel features.

14. The method of claim 13, further comprising:
acquiring data features from said retinal image; and
analyzing a set of said data features vis-à-vis a corresponding set of said blood vessel features.
